(19) United States
(12) Patent Application Publication
(10) Pub. No.: US 2023/0108443 A1
(43) Pub. Date: Apr. 6, 2023

(54) COSMETIC SKIN CONDITIONING COMPOSITION WITH HIGH UREA CONTENT

(71) Applicant: L'OREAL, Paris (FR)

(72) Inventors: Yixin YANG, Union, NJ (US); Rebecca CHEN, Princeton, NJ (US); Anne-Laure BERNARD, New York, NY (US)

(73) Assignee: L'OREAL, Paris (FR)

(21) Appl. No.: 17/953,391

(22) Filed: Sep. 27, 2022

Related U.S. Application Data
(60) Provisional application No. 63/250,586, filed on Sep. 30, 2021.

(30) Foreign Application Priority Data
Dec. 15, 2021 (FR) 2113556

(51) Int. Cl. A61K 8/42; A61K 8/44; A61K 8/37; A61K 8/34; A61Q 19/00

(52) U.S. Cl. CPC A61K 8/42; A61K 8/44; A61K 8/375; A61K 8/345; A61Q 19/007; A61K 2800/30

(57) ABSTRACT

A skin conditioning composition includes a carrier system having a water phase, the water phase including urea or a derivative thereof, present in an amount from about 20% to about 40%, by weight, based on the weight of the composition; at least one amino acid; at least one of triacetin or triethyl citrate; and, optionally, a buffer system having a pH in a range from about 4.0 to about 6.0, wherein the pH of the skin conditioning composition is less than about 7. The skin conditioning composition according carrier system may also include one more additional ingredients, each present in the water phase or in a second phase.

### RELATED APPLICATIONS

This application claims the benefit of, and priority to, U.S. Provisional Patent Application Ser. No. 63/250,586, filed on Sep. 30, 2021, entitled COSMETIC SKIN CONDITIONING COMPOSITION WITH HIGH UREA CONTENT, as well as French Application No. FR2113556, filed on Dec. 15, 2021, the entirety of which are incorporated by reference herein.

### FIELD OF TECHNOLOGY

The present disclosure is in the field of cosmetic compositions for topical application to keratinous tissue, particularly compositions that include urea in high amounts.

### BACKGROUND

Many cosmetic compositions are provided with select actives that provide any number of benefits, for example, including, but not limited to, skin desquamation (peeling). Urea is a commonly used ingredient as a humectant. High concentrations of urea for skin care are desirable for the humectant properties, including as a means to promote the stratum corneum to swell, and for stimulating skin desquamation. Urea is found in many compositions that include other skin actives. Various compositions that may include high concentrations of urea for skin care are thus desirable for the humectant properties. Such skin care compositions would beneficially include urea or a derivative thereof in a range from about 20% to 40% by weight of the composition. However, it is well known that topical preparations that contain high concentrations of urea present many challenges One challenge relates to the tendency of urea to undergo a continuous hydrolysis reaction that leads to pH increase, which can result in an increase from a pH of about 7 up to a pH of 9 or greater over the course of storage. Such pH values at or greater than a pH of 8 are much higher than that of the natural skin, which is about 5.5, and compositions with a pH in this range would likely cause skin irritation when applied. Thus, such high resultant pH renders most formulations unsuitable for use on skin, particularly in water-based compositions which don't provide protection through barrier forming oils and butters. This is particularly an issue for water-based leave-on compositions. Yet another challenge is the tendency of urea to crystalize at high concentrations and at low temperature over time. This crystallization would be expected to reduce the amount of active urea available in the usable composition, thus diminishing the overall desired efficacy of the product. A further challenge with high urea content is whitening of the skin which is not aesthetically pleasing to many consumers.

The inventors provide here a cosmetic skin care composition comprising a skin conditioning composition that is used in some embodiments for skin rejuvenation that includes use of the skin conditioning composition followed by use of a secondary composition. In some embodiments the inventors have overcome the foregoing challenges and provide a water-based skin conditioning composition that is stable from pH increase and crystallization to maintain a high amount of urea for achieving efficacy as a humectant, for promoting the stratum corneum to swell, and for stimulating skin desquamation, among other possible benefits.

### BRIEF SUMMARY OF THE INVENTION

This summary is provided to introduce a selection of concepts in a simplified form that are further described below in the detailed description of the invention. This summary is not intended to identify key features of the claimed subject matter, nor is it intended to be used as an aid in determining the scope of the claimed subject matter.

In accordance with various embodiments, provided is a the skin conditioning composition that includes a carrier system having a water phase, the water phase including urea or a derivative thereof, present in an amount from about 20% to about 40%, by weight, based on the weight of the composition; at least one amino acid; at least one of triacetin or triethyl citrate; and, optionally, a buffer system having a pH in a range from about 4.0 to about 6.0, wherein the pH of the skin conditioning composition is less than about 7. The skin conditioning composition carrier system may also include one more additional ingredients, each present in the water phase or in a second phase.

In various embodiments, the invention provides a skin conditioning composition, comprising:

urea or a derivative thereof, present in an amount from about 20% to about 40%, by weight, based on the weight of the composition; at least one amino acid; at least one of triacetin or triethyl citrate; optionally, a buffer system having a pH in a range from about 4.0 to about 6.0, wherein the pH of the skin conditioning composition is less than about 7.

In some embodiments, the composition is formulated as a leave-on product.

The skin conditioning composition according to claim 1, wherein the pH is in a range from about 4.0 to about 6.0.

In some embodiments, the at least one amnio acid is soluble in water at a pH from about 4 to about 6.

In some embodiments, the at least one amnio acid is selected from the group consisting of glycine, proline, serine, cysteine and combinations thereof.

In some embodiments, the at least one amnio acid is present in an amount from about 5% to about 9%, by weight, based on the weight of the composition.

In some embodiments, the at least one of triacetin or triethyl citrate is present in an amount from about 0.2% to about 2%%, by weight, based on the weight of the composition.

In some embodiments, the invention provides a skin conditioning composition wherein the at least one amino acid is selected from the group consisting of glycine, proline, serine, cysteine and combinations thereof and is present in an amount from about 5% to about 9%, by weight, based on the weight of the composition; and the at least one of triacetin or triethyl citrate, is present in an amount a range that includes about 0.2% to about 2%, by weight, based on the weight of the composition; and wherein the skin conditioning composition comprises a buffer system comprising one or a combination of components selected from the group consisting of citric acid, sodium citrate, sodium lactate, lactic acid, and combinations thereof, wherein the skin conditioning composition demonstrates structural and pH stability for periods of up to about 8 weeks under temperature conditions that range from 4° C. up to 45° C. and through freeze thaw cycles from −20° C. through 20° C., characterized as the absence of any or sustained visible crystallization and the stabilization of pH at or under a pH of about 7.

In some embodiments, the skin conditioning composition further comprising at least one pH adjusting reagent comprising triethanolamine.

In some embodiments, the urea or a derivative thereof, is present in an amount from greater than about 20% to about 40%, by weight, based on the weight of the composition, and further comprising at least one glycol selected from the group consisting of propylene glycol, glycerin, and combinations thereof.

In some embodiments, the skin conditioning composition comprises propylene glycol and excludes glycerin.

In some embodiments, the invention provides a skin conditioning composition, comprising: urea present from greater than 20% to about 40%; at least one amino acid, the at least one amino acid comprising glycine present from about 5% to about 9%; at least one of triacetin or triethyl citrate, present from about 0.2% to about 2%; a buffer system having a pH in a range from about 4.0 to about 6.0; at least one pH adjusting reagent, comprising triethanolamine; and at least one glycol selected from the group consisting of propylene glycol, glycerin, and combinations thereof, wherein all amounts are by weight, based on the weight of the composition, and wherein the pH is from about 4.0 to about 6.0, and wherein the skin conditioning composition demonstrates structural and pH stability for periods of up to about 8 weeks under temperature conditions that range from 4° C. up to 45° C. and through freeze thaw cycles from −20° C. through 20° C., characterized as the absence of any or sustained visible crystallization and the stabilization of pH at or under a pH of about 7.

In some embodiments, the skin conditioning composition comprises propylene glycol and excludes glycerin.

In some embodiments, the buffer system comprises one or a combination of components selected from the group consisting of citric acid, sodium citrate, sodium lactate, lactic acid, and combinations thereof.

In some embodiments, the buffer system comprises sodium lactate and lactic acid.

In some embodiments, the invention provides a skin conditioning composition, comprising: a carrier system comprising a water phase, the water phase comprising urea or a derivative thereof, present in an amount from about 20% to about 40%, by weight, based on the weight of the composition; at least one amino acid; at least one of triacetin or triethyl citrate; and, optionally, a buffer system having a pH in a range from about 4.0 to about 6.0, wherein the pH of The skin conditioning composition is less than about 7.

In some embodiments, the carrier system further comprises one more additional ingredients, each present in the water phase or in a second phase.

The skin conditioning composition according to claim 17, wherein the second phase is an oily phase.

In some embodiments, the one or more additional ingredients includes one or more of an additional ingredient selected from the group consisting of a skin active compound, a humectant in addition to any glycol, an oil, a surfactant, a thickener, a preservative, and combinations thereof.

In some embodiments, the cosmetic composition is one of a single phase solution comprising the water phase, a multi-phase solution comprising at least one additional phase, or an emulsion comprising at least one oily phase.

In some embodiments, the skin conditioning composition is a product for keratinous tissue selected from a water toner, a gel, a mask, a micellar water, a cream, a lotion, a cleanser, or a make-up remover.

In some embodiments, the skin conditioning composition is a leave-on formulation.

In another aspect, the present disclosure provides a method of contacting a portion of skin with cosmetic skin conditioning composition with or without an implement or an applicator. The methods may further include application of a complementary composition, before or after application of the cosmetic skin conditioning composition.

The detailed description set forth below in connection with the appended drawings, where like numerals reference like elements, is intended as a description of various embodiments of the disclosed subject matter and is not intended to represent the only embodiments. The embodiments described in this disclosure are provided merely as examples or illustrations and should not be construed as preferred or advantageous over other embodiments. The illustrative examples provided herein are not intended to be exhaustive or to limit the claimed subject matter to the exact forms disclosed.

### DETAILED DESCRIPTION OF THE INVENTION

The cosmetic composition of the present invention may be used for the production of cosmetic preparations, or dermatological preparations, more particularly topical treatment preparations, that may be formulated as single-phase water-based solution compositions, cosmetic serums, or aerosols, for example. Topical application to a surface may be a surface such as the mucus membrane or the skin, for example.

As used herein, the term “skin conditioning” in reference to the inventive cosmetic skin conditioning composition means acting as a humectant. In some embodiments, skin conditioning can also include promoting the stratum corneum of skin to swell to thereby stimulate the skin to be responsive to exfoliation.

“Cosmetically acceptable” means compatible with any keratinous substrate. For example, “cosmetically acceptable carrier” means a carrier that is compatible with any keratinous substrate.

The term “stable” as used in reference to the structural or solubility state of the ingredients of the cosmetic skin conditioning composition means that the cosmetic skin conditioning composition lacks crystal formation that is visible to the naked eye, and does not demonstrate visually perceptible crystal formation over a period of time, for example for one, two or four or five days, or for a period of one or more weeks, and for example up to eight (8) weeks, and wherein when crystals are visualized at a temperature at or below 4° C., such crystals disappear with or without mild agitation within 24 hours of reaching an ambient temperature from about 25° C. up to about 37° C. and after freeze thaw cycles from −20° C. through 20° C. The term “stable” as used in reference to the pH the cosmetic skin conditioning composition means that the pH does not increase appreciably over time, for example as described in the examples herein.

The term “leave-on” as used herein refers to a composition which is applied and is not removed by wiping or rinsing with water.

Applicants have unexpectedly found that skin conditioning composition demonstrates structural and pH stability for periods of up to about 8 weeks under temperature conditions that range from 4° C. up to 45° C. and through freeze thaw cycles from −20° C. through 20° C. These stabilities are characterized as the absence of any or sustained visible crystallization and the stabilization of pH at or under a pH of about 7, in particular in embodiments that include greater than 20% and up to at least 40% urea. Examples provided herein demonstrate these and other aspects of the unexpected properties of the skin conditioning composition.

In various embodiments, the cosmetic skin conditioning composition is formed by providing the carrier system having a water-phase that comprises water. In various embodiments, the carrier system may further comprise one more additional ingredients, each present in the water phase or in another phase, such as an oily phase. In various embodiments, the one or more additional ingredients includes one or more of an additional ingredient selected from the group consisting of a skin active compound, humectant in addition to any glycol, a preservative, a surfactant, a thickener, and combinations thereof. In some embodiments, the cosmetic skin conditioning composition may include one or more of any one of the additional ingredients. In some embodiments, the cosmetic skin conditioning composition may also include fragrances, pH adjusters, other cosmetically acceptable additives, or a combination thereof.

The cosmetic skin conditioning composition may be any suitable form, such as, but not limited to, a single phase solution comprising the water phase, a multi-phase solution comprising at least one water phase, an emulsion comprising a water phase and an oily phase, such as, but not limited to, a water-in-oil emulsion, an oil-in-water emulsion, or a silicone-in-water emulsion. The cosmetic skin conditioning composition may be provided in a product form for application to keratinous tissue, such as, but not limited to, a skin care product or a hair care product, and may be a product form such as, but not limited to, a micellar water, a cream or lotion, a toner, a cleanser, or a make-up remover. The cosmetic skin conditioning composition in any of the product forms may be either a leave-on or a rinse-off formulation. In certain embodiments, as exemplified herein, the cosmetic skin conditioning composition is a leave-on formulation.

As further described herein below, the skin conditioning composition includes a carrier system having a water phase, the water phase including urea or a derivative thereof, present in an amount from about 20% to about 40%, by weight, based on the weight of the composition; at least one amino acid; at least one of triacetin or triethyl citrate; and, optionally, a buffer system having a pH in a range from about 4.0 to about 6.0, wherein the pH of the skin conditioning composition is less than about 7. The skin conditioning composition carrier system may also include one more additional ingredients, each present in the water phase or in a second phase.

Urea

cosmetic skin conditioning composition urea is present in an amount sufficient to enhance penetration of the active ingredient(s).

In some embodiments, urea may be provided as one of urea, or a urea derivative, for example, hydroxyethyl urea (HEU), wherein urea has a formula (I) and HEU has a formula (II) as shown below.

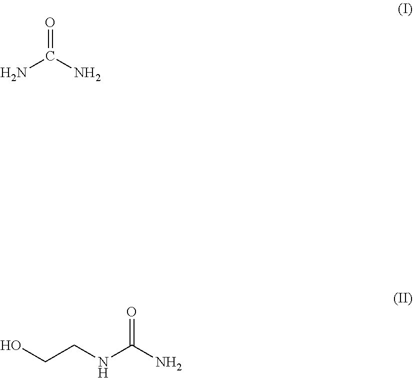

Molecular weight information for each of these is shown in Table 1.

TABLE 1

| | Molecular Weight | wt % | Mol/L* (at 1 g/L) |
|---|---|---|---|
| HEU | 104 | 40% | 3.85 |
| Urea | 60 | 40% | 6.67 |
| HEU | 104 | 23% | 2.21 |
| Urea | 60 | 23% | 3.88 |

Generally, a urea or a derivative thereof is selected for its beneficial effects to the skin, which effect is to be enhanced by exposure to urea of the viable epidermis and/or dermis, which underlies the stratum corneum, wherein a urea derivative can be described by the general formula (III):

R¹R²N—C(═O)—NR³R⁴ (III)

wherein R1, R2, R3 and R4 each represent, independently of each other, a hydrogen atom, a C1-C4 alkyl group or a C2-C6 hydroxyalkyl group which optionally contains from 1 to 5 hydroxyl groups, where at least one of the radicals R1 to R4 represents a hydroxyalkyl group, and their salts, their solvates and theft isomers in a composition containing a physiologically acceptable medium, as agent intended for stimulating desquamation of the skin and/or the mucous membranes and/or the superficial body growths. As described in U.S. Pat. No. 5,799,008, such urea derivatives have a remarkable effect on several mechanisms involved in desquamation, thus allowing a complete and natural action of stimulating this process, and the application in all cases where it is desirable to promote and/or accelerate the removal of the dead layers of the epidermis. For compounds of formula (III):

Preferably, R, denotes a C2-C6 hydroxyl group, and R2, R3 and R4 denote, independently of each other, a hydrogen atom or a C1-C4 alkyl group;

Preferably, R1 denotes a C2-C6 hydroxyalkyl group comprising from 1 to 5 hydroxyl groups, in particular 1 hydroxyl group, and R2, R3 and R4 denote a hydrogen atom;

More preferably, R1 denotes a C2-C4 hydroxyalkyl group comprising 1 hydroxyl group, and R2, R3 and R4 denote a hydrogen atom.

Among the alkyl groups, there may be mentioned in particular methyl, ethyl, n-propyl, isopropyl, n-butyl, isobutyl and tort-butyl groups.

Among the hydroxyalkyl groups, those containing a single hydroxyl group are preferred, and in particular the hydroxyethyl, hydroxypropyl, hydroxybutyl, hydroxypentyl and hydroxyhexyl groups, are preferred.

Among the salts, there may be mentioned in particular the salts of inorganic acids, such as sulphuric acid, hydrochloric acid, hydrobromic acid, hydriodic acid, phosphoric acid and boric acid. There may also be mentioned the salts of organic acids, which may contain one or more carboxylic, sulphonic or phosphoric acid groups. They may be linear, branched or cyclic aliphatic acids or aromatic acids. These acids may additionally contain one or more heteroatoms chosen from O and N, for example in the form of hydroxyl groups. There may be mentioned in particular propionic acid, acetic acid, terephthalic acid, citric acid and tartaric acid.

The expression solvate is understood to mean a stoichiometric mixture of the said compound of formula fill) with one or more molecules of water or of organic solvent, such a mixture typically being obtained from the synthesis of the compound of formula (III).

Embodiments of formula (III), there may be mentioned N-(2-hydroxyethyl)urea; N-(2-hydroxypropyl)-urea; N-(3-hydroxypropyl)urea; N-(2,3-dihydroxypropyl)urea; N-(2,3,4,5,6-pentahydroxyhexyl)urea; N-methyl-N-(1,3,4,5,6-pentahydroxy-2-hexyl)urea; N-methyl-N′-(1-hydroxy-2-methyl-2-propyl)urea; N-(1-hydroxy-2-methyl-2-propy)urea; N-(1,3-dihydroxy-2-propyl)urea; N-(tris-hydroxymethylmethyl)urea; N-ethyl-N′-(2-hydroxyethyl)urea; N,N-bis(2-hydroxyethyl)urea; N,N′-bis(2-hydroxyethyl)urea; N,N′-bis(2-hydroxypropyl)urea; N,N′-bis(2-hydroxypropyl)urea; N,N-bis(2-hydroxyethyl)-N′-propylurea; N,N-bis(2-hydroxypropyl)-N′-(2-hydroxy-ethyl)urea; N-tert-butyl-N′-(2-hydroxyethyl)-N′-(2-hydroxypropyl)urea; N-(1,3-dihydroxy-2-propyl)N′-(2-hydroxyethyl)urea; N,N-bis(2-hydroxyethyl)-N′,N′-dimethylurea; N,N,N′,N′-tetrakis(2-hydroxyethyl)urea; N′,N′-bis(2-hydroxyethyl)-N′,N′-bis(2-hydroxypropyl)-urea; salts and solvates thereof, and mixtures thereof.

In accordance with the various embodiments total amount of the urea or derivative thereof is in a range from about 20% to about 40%, or from about 20% to about 35%, or from about 25% to about 30%, or from about 25% to about 40%, or from about 30% to about 35% or any suitable combination, sub-combination, range, or sub-range thereof by weight, based on the weight of the composition. One of ordinary skill in the art, however, will appreciate that other ranges are within the scope of the invention.

Thus, the urea or derivative thereof, is present, by weight, based on the total weight of the composition, from about 20, 21, 22, 23, 24, 25,26,27,28, 29, 30, 31,32, 33, 34, 35,36,37, 38, 39 to about 40 weight percent, including increments and ranges therein and there between.

Amino Acid

In accordance with the various embodiments, the cosmetic skin conditioning composition includes at least one or more amino acids. In some embodiments the at least one amino acid may be selected from the group consisting of naturally occurring amino acids. In some embodiments the at least one amino acid may be selected from glycine, proline, serine and cysteine. And in some embodiments the at least one amino acid comprises glycine.

As non-limiting examples, the amino acids that may be used may be of natural or synthetic origin, in L, D, or racemic form, and comprise at least one acid function chosen from, for instance, carboxylic acid, sulfonic acid, phosphonic acid, and phosphoric acid functions. The amino acids may be in their neutral or ionic form. Polymeric forms are also useful, such as poly arginine, poly lysine, etc. Additional non-limiting examples include basic amino acids comprising an additional amine function optionally included in a ring or in a ureido function. In some cases, amino acids that may be used include, but are not limited to, aspartic acid, glutamic acid, alanine, arginine, ornithine, citrulline, asparagine, carnitine, cysteine, glutamine, glycine, histidine, lysine, isoleucine, leucine, methionine, N-phenylalanine, proline, serine, taurine, threonine, tryptophan, tyrosine, ornithine, citrulline, and valine.

In accordance with the various embodiments, the amount of amnio acid present in a composition according to the disclosure can range from about 2% to about 10%, or from about 3% to about 9%, or from about 4% to about 8%, or from about 3% to about 5%, or any suitable combination, sub-combination, range, or sub-range thereof by weight, based on the weight of the composition. In some embodiments according to the disclosure, the composition includes at least about 3%, or at least about 5% of the at least one amino acid. In some embodiments according to the disclosure, the composition includes not more than about 9% of the at least one amino acid. One of ordinary skill in the art, however, will appreciate that other ranges are within the scope of the invention. In some embodiments, two or more alkaline boosters are present.

Thus, any one of or a combination of the amino acid may be present, by weight, based on the total weight of the composition, or from about 2.0, 2.5, 3.0, 3.5, 4.0, 4.5, 5.0, 5.5, 6.0, 6.5, 7.0, 7.5, 8.0, 8.5, 9.0, 9.5 to about 10.0 weight percent, including increments and ranges therein and there between.

Triacetin/Triethyl Citrate

In accordance with the various embodiments, the cosmetic skin conditioning composition includes at least one or more of triacetin or triethyl citrate.

In accordance with the various embodiments, the amount of triacetin or triethyl citrate present in a composition according to the disclosure can range from about 0.2% to about 2%, or from about 0.3% to about 1.5%, or from about 0.5% to about 1.2%, or from about 0.7% to about 1%, or any suitable combination, sub-combination, range, or sub-range thereof by weight, based on the weight of the cosmetic skin conditioning composition. In some embodiments according to the disclosure, the cosmetic skin conditioning composition includes at least about 0.2%, or at least about 0.7% of the triacetin or triethyl citrate. In some embodiments according to the disclosure, the cosmetic skin conditioning composition includes not more than about 1% of the triacetin or triethyl citrate. One of ordinary skill in the art, however, will appreciate that other ranges are within the scope of the invention. In some embodiments, two or more alkaline boosters are present.

Thus, any one of or a combination of the triacetin or triethyl citrate may be present, by weight, based on the total weight of the cosmetic skin conditioning composition, or from about 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, to about 2 weight percent, including increments and ranges therein and there between.

Buffer System

In accordance with the various embodiments, the cosmetic skin conditioning composition may include a buffer system. In some embodiments, the buffer system comprises one or a combination of components selected from the group consisting of citric acid, sodium citrate, sodium lactate, lactic acid, and combinations thereof. In some particular embodiments, the buffer system comprises sodium lactate, lactic acid, ammonium lactate, and combinations thereof. In some particular embodiments, the buffer system comprises citric acid, sodium citrate, and combinations thereof.

More generally, the buffer system is selected from conventional buffer systems commonly used for compositions used on keratinous tissue, particularly skin. Buffer systems may include one or a combination of acidifying agents and alkalifying agents. Acidifying agents include, for example, organic or inorganic acids such as hydrochloric acid, orthophosphoric acid, sulphuric acid, carboxylic acids such as acetic acid, tartaric acid, citric acid and lactic acid, and sulphonic acids. Alkalifying agents include, for example, aqueous ammonia, alkali metal carbonates, alkanolamines such as mono-, di- and triethanolamines and their derivatives, sodium hydroxide or potassium hydroxide.

In accordance with the various embodiments, the amount of buffer components, when present in a cosmetic skin conditioning composition according to the disclosure, generally conform to those employed conventionally. According to the disclosure, a buffer system comprising, for example, sodium lactate, lactic acid, ammonium lactate, and combinations thereof, includes each of the buffer system components present in a range from about 0.1% to about 3%, or from about 0.25% to about 2.5%, or from a total of about 0.1% to about 6%, or from about 0.25% to about 5.25%, or any suitable combination, sub-combination, range, or sub-range thereof by weight, based on the weight of the cosmetic skin conditioning composition. More generally, in some embodiments according to the disclosure, the cosmetic skin conditioning composition includes each one or more buffer components present in a range from about 0.1% to about 15%. One of ordinary skill in the art, however, will appreciate that other ranges are within the scope of the invention. In some embodiments, two or more alkaline boosters are present.

Thus, any one of or a combination of buffer system components may be present, by weight, based on the total weight of the cosmetic skin conditioning composition, or from about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 3, 4, 5, 6, 7, 8, 9, 10, 11,12, 13, 14, to about 15 weight percent, including increments and ranges therein and there between.

Glycol

In accordance with the various embodiments, the cosmetic skin conditioning composition may include at least one alkanol, and particularly at least one glycol. In some embodiments, the cosmetic skin conditioning comprises one or a combination of alkanols (polyhydric alcohols such as glycols and polyols). In some embodiments, the cosmetic skin conditioning comprises one or a combination of propylene glycol, glycerin, propanediol, butylene glycol, dipropylene glycol, pentylene glycol, hexylene glycol, ethoxydiglycol, and combinations thereof. In some particular embodiments, the cosmetic skin conditioning may comprise at least propylene glycol, with or without glycerin.

More generally, the cosmetic skin conditioning may comprise one or a combination of the 1,2,6-hexanetriol, trimethylolpropane, ethylene glycol, diethylene glycol, butylne glycol, hexylene glycol, triethylene glycol, tetraethylene glycol, pentaethylene glycol, dipropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, or mixtures thereof.

In accordance with the various embodiments, the amount of the at least one glycol present in the cosmetic skin conditioning composition can range from about 1% to about 15%, or from about 2% to about 12%, or from about 3% to about 10%, or from about 3.5% to about 5%, or any suitable combination, sub-combination, range, or sub-range thereof by weight, based on the weight of the cosmetic skin conditioning composition. One of ordinary skill in the art, however, will appreciate that other ranges are within the scope of the invention.

Thus, any one of or a combination of glycol, when present, may be present, by weight, based on the total weight of the cosmetic skin conditioning composition, is from about 1, 1.1, 1.2, 1.3, 1.4, 1.5, 1.6, 1.7, 1.8, 1.9, 2.0, 2.5, 3.0, 3.5, 4.0, 4.5, 5.0, 5.5, 6.0, 6.5, 7.0, 7.5, 8.0, 8.5, 9.0, 9.5, 10, 10.5, 11, 11.5, 12, 12.5 13, 13.5, 14, 14.5, to about 15 percent, by weight, based on the weight of the cosmetic skin conditioning composition, including increments and ranges therein and there between.

Carrier System

In various embodiments, the cosmetic skin conditioning composition is formed by providing a carrier system having a water-phase that comprises water. The urea, amino acid, triacetin/Triethyl citrate, and buffer system components are typically preferentially in the water phase. As described herein below, the cosmetic skin conditioning composition may include other ingredients, and the carrier system may include other phases, including one or more additional water and/or oil phases.

Water Phase/Water

In various embodiments, the cosmetic skin condition composition includes at least one water phase that includes water.

The cosmetic skin conditioning composition comprises from about 40% to about 70% by weight of water, with respect to the total weight of the cosmetic skin conditioning composition. In some embodiments, the amount of water in the cosmetic skin conditioning composition can range from about 40% to about 70%, or from about 45% to about 65%, or from about 50% to about 60%, or any suitable combination, sub-combination, range, or sub-range thereof by weight, based on the weight of the cosmetic skin conditioning composition.

The pH of the cosmetic skin conditioning composition is in the range from and including about 4 to about 6, and in some embodiments is one of between 5.5 and 6.5, or between 4 and 5, or between 5 and 6, or up to about 7. The pH can be adjusted to the desired value by addition of a base (organic or inorganic) to the cosmetic skin conditioning composition, for example ammonia or a primary, secondary or tertiary (poly)amine, such as monoethanolamine, diethanolamine, triethanolamine, isopropanolamine or 1,3-propanediamine, or alternatively by addition of an inorganic or organic acid, for example a carboxylic acid, such as, for example, citric acid.

Thus, water may be present by weight, based on the total weight of the cosmetic skin conditioning composition, or from about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, to about 70 weight percent, including increments and ranges therein and there between.

Optional Components

Cosmetic Oils/Emollients

In accordance with some embodiments, the cosmetic skin conditioning composition may comprise at least one oily phase which may include any one or a combination of cosmetic oils. In some embodiments, the oil is generally immiscible in water. The oil may be selected from hydrocarbons, silicones, fatty alcohols, glycols, and vegetable oils. The oil may include one or a combination of polar and non-polar oil. In some embodiments, the oil may be chosen from hydrocarbon-based oils from plants or of plant origin, mineral oil, ester oils, fatty alcohols containing from 12 to 26 carbon atoms, fatty acids containing from 12 to 26 carbon atoms and vinylpyrrolidone copolymers, and combinations thereof.

In some particular embodiments, the oil may be selected from the group consisting of, coco-caprylate/caprate, dicaprylyl carbonate, dicaprylyl ether, isopropyl palmitate, and combinations thereof.

The term “silicone oil” relates to oil comprising at least one silicon atom, and especially at least one Si—O group. The term “fluoro oil” relates to oil comprising at least one fluorine atom. The term “hydrocarbon-based oil” relates to oil comprising mainly hydrogen and carbon atoms. Hydrocarbon-based oil may be animal hydrocarbon-based oil, plant hydrocarbon-based oil, mineral hydrocarbon-based oil, or a synthetic hydrocarbon-based oil. Further, suitable oil may be a mineral hydrocarbon-based oil, a plant hydrocarbon-based oil, or a synthetic hydrocarbon-based oil.

Silicone Oils

The cosmetic skin conditioning composition may comprise one or more silicone oils. Non-limiting examples of silicone oils include dimethicone, cyclomethicone, polysilicone-11, phenyl trimethicone, trimethylsilylamodimethicone, and stearoxytrimethylsilane. In some cases, the cosmetic skin conditioning composition includes dimethicone, and optionally additional oils, including additional silicone oils. Typically, the one or more silicone oils is a non-volatile silicon oil. In some embodiments, the silicone oil is polydimethylsiloxanes (PDMSs), polydimethylsiloxanes comprising alkyl or alkoxy groups which are pendent and/or at the end of the silicone chain, which groups each contain from 2 to 24 carbon atoms, or phenyl silicones, such as phenyl trimethicones, phenyl dimethicones, phenyl(trimethylsiloxy)diphenylsiloxanes, diphenyl dimethicones, diphenyl(methyldiphenyl)trisiloxanes or (2-phenylethyl)trimethylsiloxysilicates.

Other examples of silicone oils that may be mentioned include volatile linear or cyclic silicone oils, especially those with a viscosity 8 centistokes (8×106 m2/s) and especially containing from 2 to 7 silicon atoms, these silicones optionally comprising alkyl or alkoxy groups containing from 1 to 10 carbon atoms. As volatile silicone oils that may be used in the invention, mention may be made especially of octamethylcyclotetrasiloxane, decamethylcyclopentasiloxane, dodecamethylcyclohexasiloxane, hepta methylhexyltrisiloxane, heptamethyloctyltrisiloxane, hexamethyldisiloxane, octamethyltrisiloxane, decamethyltetrasiloxane and dodecamethylpentasiloxane, and mixtures thereof.

Fluoro Oils

The cosmetic skin conditioning composition may comprise one or more fluoro oils. For example, the one or more fluoro oil may be selected from the group consisting of perfluoromethylcyclopentane, perfluoro-1,3-dimethylcyclohexane, dodecafluoropentane, tetradecafluorohexane, bromoperfluorooctyl, nonafluoromethoxybutane, nonafluoroethoxyisobutane and 4-trifluoromethylperfluoromorpholine. Volatile fluoro oils, such as nonafluoromethoxybutane, decafluoropentane, tetradecafluorohexane, dodecafluoropentane, may also be used.

Hydrocarbon-Based Oils

The cosmetic skin conditioning composition may comprise one or more hydrocarbon-based oils. For example, the hydrocarbon-based oil may be a saturated hydrocarbon, an unsaturated hydrocarbon, lipids, triglycerides, a natural oil, and/or a synthetic oil. In some embodiments, the cosmetic skin conditioning composition may include a synthetic oil selected from the group consisting of hydrogenated polyisobutene and hydrogenated polydecene. A hydrocarbon-based oil may be a non-volatile hydrocarbon-based, such as:

(i) hydrocarbon-based oils of plant origin, such as glyceride triesters, which are generally triesters of fatty acids and of glycerol, the fatty acids of which can have varied chain lengths from C4 to C24, it being possible for these chains to be saturated or unsaturated and linear or branched; these oils are in particular wheat germ oil, sunflower oil, grape seed oil, sesame oil, corn oil, apricot oil, castor oil, rhea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin seed oil, marrow oil, blackcurrant oil, evening primrose oil, millet oil, barley oil, quinoa oil, rye oil, safflower oil, candlenut oil, passionflower oil, and musk rose oil.

(ii) synthetic ethers containing from 10 to 40 carbon atoms;

(iii) linear or branched hydrocarbons of mineral or synthetic origin, such as petroleum jelly, polydecenes, hydrogenated polyisobutene such as Parleam, and 40 squalane;

(iv) synthetic esters, for instance oils of formula RCOOR′ in which R represents a linear or branched fatty acid residue containing from 1 to 40 carbon atoms and R′ represents a hydrocarbon-based chain that is especially branched, containing from 1 to 40 carbon atoms on condition that R+R′ is ÿ 10, for instance Purcellin oil (cetearyl octanoate), isopropyl myristate, isopropyl palmitate, C12-C15 alkyl benzoate, such as the product sold under the trade name Finsolv TN® or Witconol TN® by Witco or Tegosoft TN® by Evonik Goldschmidt, 2-ethylphenyl benzoate, such as the commercial product sold under the name X-Tend 226 by ISP, isopropyl lanolate, hexyl laurate, diisopropyl adipate, isononyl isononanoate, oley erucate, 2-ethylhexyl palmitate, isostearyl isostearate, diisopropyl sebacate, such as the product sold under the name of “Dub Dis” by Stearinerie Dubois, octanoates, decanoates or ricinoleates of alcohols or polyalcohols, such as propylene glycol dioctanoate; hydroxylated esters, such as isostearyl lactate or diisostearyl malate; and pentaerythritol esters; citrates or tartrates, such as di(linear C12-C13 alkyl) tartrates, such as those sold under the name Cosmacol ETI® by Enichem Augusta lndustriale, and also di(linear C14-C15 alkyl) tartrates, such as those sold under the name Cosmacol ETL® by the same company; or acetates;

(v) fatty alcohols that are liquid at room temperature, containing a branched and/or unsaturated carbon-based chain containing from 12 to 26 carbon atoms, for instance octyldodecanol, isostearyl alcohol, oley alcohol, 2-hexyldecanol, 2-butyloctanol or 2-undecylpentadecanol;

(vi) higher fatty acids, such as oleic acid, linoleic acid, or linolenic acid;

(vii) carbonates, such as dicaprylyl carbonate, such as the product sold under the name Cetiol CC™ by Cognis;

(viii) fatty amides, such as isopropyl N-lauroyl sarcosinate, such as the product sold under the trade name Eldew SL 205™ from Ajinomoto; and

(ix) essential oils selected from the group consisting of sunflower oil, sesame oil, peppermint oil, macadamia nut oil, tea tree oil, evening primrose oil, sage oil, rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, and ylang ylang.

Hydrocarbon-based oils may be glyceride triesters and in particular to caprylic/capric acid triglycerides, synthetic esters and in particular isononyl isononanoate, oley erucate, C12-C15 alkyl benzoate, 2-ethylphenyl benzoate and fatty alcohols, such as octyldodecanol. As volatile hydrocarbon-based oils, mention is made of hydrocarbon-based oils containing from 8 to 16 carbon atoms and in particular of branched C8-C16 alkanes, such as C8-C16 isoalkanes of petroleum origin (also known as isoparaffins), such as isododecane (also known as 2,2,4,4,6-pentamethylheptane), isodecane or isohexadecane, the oils sold under the lsopar or Permethyl trade names, branched C C8-C16 esters, and isohexyl neopentanoate.

In some embodiments, the cosmetic skin conditioning composition may comprise one or more oils such as from those described herein above, and from oils that may be selected from branched or linear, liquid alkane with carbon chain length of C11 to C20. In various embodiments, liquid alkanes may be selected from those with a carbon chain length of from C11 to C20. The liquid alkanes may be selected from those with a carbon chain length of from C11 to C20, or from C15 to C19, or one of C11, C12, C13, C14, C15, C16, C17, C18 to C19. In some embodiments, suitable liquid alkanes that may be used according to the disclosure include hydrocarbon-based oils containing from 8 to 16 carbon atoms, and especially branched C8-C16 alkanes such as C8-C16 isoalkanes.

In some embodiments, the cosmetic skin conditioning composition may comprise one or more oils selected from polar emollients selected from esters, triglycerides, ethers, carbonates, alcohols, oils, butters, fatty acids, and their combinations thereof. In various embodiments, the polar emollients may be selected from those with a molecular weight of 400 g/mol or less. More, generally, the polar emollient may have a molecular weight in the range from about 50 g/mol % to about 350 g/mol.

In some embodiments, the cosmetic skin conditioning composition may comprise polar emollients that include those derived from C12-C50 fatty acids, preferably C16-C22 saturated fatty acids, and monohydric alcohols. In some embodiments, such esters may be chosen from isopropyl myristate, methyl palmitate, isopropyl laurate, isopropyl palmitate, ethylhexyl palmitate, ethylhexyl laurate, ethylhexyl oleate, ethylhexyl isononanoate, myristyl myristate, 2-ethylhexyl caprate/caprylate (or octyl caprate/caprylate), 2-ethylhexyl palmitate, isostearyl neopentanoate, isononyl isononanoate, hexyl laurate, esters of lactic acid and of fatty alcohols comprising 12 or 13 carbon atoms, dicaprylyl carbonate and their mixtures.

The one or more oil, when present, alone or in combination as a blend of oils, may be present in the cosmetic skin conditioning composition from about 0.0001% to about 20% or from about 0.001% to about 0.0010%, or from about 0.003% to about 0.004%, or from about 0.01% to about 0.1%, or from about 0.1% to about 10%, or from about 0.5% to about 20%, or from about 1% to about 10%, or from about 5% to about 10%, or from about 2% to about 7%, or from about 0.5% to about 2%, or from about 0.008% to about 0.01%, or from about 0.1% to about 0.2%, or from about 0.5% to about 2%, or from about or any suitable combination, sub-combination, range, or sub-range thereof by weight, based on the weight of the cosmetic skin conditioning composition. One of ordinary skill in the art, however, will appreciate that other ranges are within the scope of the invention. In some embodiments, the cosmetic skin conditioning composition includes more than one oil, each oil present in an amount as set forth herein above, wherein each different oil (such as, for example, plant oils and extracts with oils) may be present within one of the ranges selected from the ranges set forth herein above.

Thus, each of the at least one oil or combination of oils, if present, is present by weight, based on the total weight of the cosmetic skin conditioning composition, from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19 to about 20 percent, including increments and ranges therein and there between.

Surfactant

In accordance with the disclosure, various non-limiting embodiments of the cosmetic skin conditioning composition may optionally include at least one surfactant. And in accordance with the disclosure, various non-limiting embodiments of the cosmetic skin conditioning composition that include an oil, for example, but not limited to, a Vitamin E component, such as tocopherol, may optionally include at least one surfactant. In some embodiments, the cosmetic skin conditioning composition comprises no oil. In some embodiments, the cosmetic skin conditioning composition comprises no surfactant. In some embodiments, the cosmetic skin conditioning composition comprises at least one oil, for example, tocopherol, with at least one surfactant. In some embodiments, the cosmetic skin conditioning composition comprises at least one oil with more than one surfactant. In some embodiments, the cosmetic skin conditioning composition comprises at least one oil without any surfactants, wherein the total amount of oil present does not inhibit forming the cosmetic skin conditioning composition as a clear, single phase solution.

The at least one surfactant may be an nonionic, cationic, anionic, or a zwitterionic surfactant. The at least one surfactant may be selected from the group consisting of polyoxyethylene sorbitan monolaureate, laureth-23, polyoxyethylated octyl phenol, 3-((3-cholamidopropyl) dimethylammonio)-1 propane sulfonate, sodium dilauramidoglutamide lysine, cholate, deoxycholate, sodium dodecylsulfate, TWEEN-80, and combinations thereof. The at least one surfactant may exclude esters. The at least one surfactant may be present in the cosmetic skin conditioning composition in a range from about 1% to about 5%, based on the weight of the cosmetic skin conditioning composition.

In some particular embodiments the at least one surfactant, when present, comprises one or a combination of polyoxyethylene sorbitan monolaureate and laureth-23.

In various embodiments, the at least one surfactant, when present, may be present from about 1% to about 5% by weight of the cosmetic skin conditioning composition, and in some embodiments, from about 1% to about 2% , and in some embodiments, from about 3% to about 6%, and in some embodiments, from about 3% to about 4.5%, or any suitable combination, sub-combination, range, or sub-range thereof by weight, based on the weight of the cosmetic skin conditioning composition. In some embodiments, laureth-23 may be present from about 1% to about 5% by weight of the cosmetic skin conditioning composition, and in some embodiments, from about 3% to about 4.5%, or any suitable combination, sub-combination, range, or sub-range thereof by weight, based on the weight of the cosmetic skin conditioning composition. In some embodiments, polyoxyethylene sorbitan monolaureate may be present from about 1% to about 5% by weight of the cosmetic skin conditioning composition, and in some embodiments, from about 1% to about 2%, or any suitable combination, sub-combination, range, or sub-range thereof by weight, based on the weight of the cosmetic skin conditioning composition. In some embodiments, the cosmetic skin conditioning composition comprises more than one surfactant. One of ordinary skill in the art, however, will appreciate that other ranges are within the scope of the invention.

Thus, in various embodiments, when present, each of the at least one surfactant may be present in a composition according to the disclosure from about 1, 2, 3, 4, to about 5 percent, by weight, including increments and ranges therein and there between.

Polymeric Thickener

In accordance with the disclosure, various non-limiting embodiments of the cosmetic skin conditioning composition may optionally include one or more polymeric thickeners. In some embodiments, the one or more thickener may be selected from one or more of natural gums and synthetic polymers, for example, the thickener may be selected from the group consisting of starches (corn, rice, tapioca, potato), gums (xanthan carrageenan, gellan, sclerotium, tarabiotech fermentation). In some particular embodiments, the thickener may be selected from acrylates/C10-30 alkyl acrylate crosspolymer, carbomer, xanthan gum, hydroxypropyl guar, ceratonia siliqua (carob) gum, ammonium polyacryloyldimethyl taurate, ammonium acryloyldimethyltaurate/steareth−25 methacrylate crosspolymer, and polyacrylate crosspolymer-6.

In some embodiments, the polymeric thickener may be one of carbomer, acrylates/C10-30 alkyl acrylate crosspolymer, polyacrylate crosspolymer-6, microcrystalline cellulose (and) cellulose gum, xanthan gum, sodium carboxymethyl starch, sclerotium gum (and) xanthan gum, xanthan gum (and) ceratonia siliqua (carob) gum (50/50), dehydroxanthan gum, hydroxypropyl starch phosphate, sclerotium gum (and) xanthan gum (75/25), sclerotium gum, xanthan gum (and) sclerotium gum (and) Lecithin (and) pullulan, or combinations thereof.

In some embodiments the cosmetic skin conditioning composition may comprise two or more polymeric thickeners. The amount of each of the at least one polymeric thickener, when present, may be present in the cosmetic skin conditioning composition in a range of from about 0.01% to about 2%, or from about 0.01% to about 1.5%, or from about 0.3% to about 1.2%, or any suitable combination, sub-combination, range, or sub-range thereof by weight, based on the weight of the cosmetic skin conditioning composition. One of ordinary skill in the art, however, will appreciate that other ranges are within the scope of the invention.

In some embodiments, the total amount of polymeric thickener in the cosmetic skin conditioning composition, when present, is present from about 0.01% to about 5%, or from about 0.02% to about 2%, or from about 0.03% to about 1.5%, or from about 0.1% to about 0.2%, or any suitable combination, sub-combination, range, or sub-range thereof by weight, based on the weight of the cosmetic skin conditioning composition. One of ordinary skill in the art, however, will appreciate that other ranges are within the scope of the invention.

Thus, one or more polymeric thickener, when present, is present by weight, based on the total weight of the cosmetic skin conditioning composition, from about 0.01, 0.02, 0.03, 0.04, 0.05, 0.06, 0.07, 0.08, 0.09, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5 to about 5.0 percent, including increments and ranges therein and there between.

Preservatives

In accordance with the disclosure, various non-limiting embodiments of the cosmetic skin conditioning composition may optionally include one or more preservatives and/or antimicrobials. Any preservative commonly used in cosmetic formulations is an acceptable preservative for the cosmetic skin conditioning compositions herein, such as phenoxyethanol, salicylic acid, members from the paraben family such as the methyl, ethyl, propyl, butyl or isobutyl parabens, 4-hydroxy benzoic acid, benzoic acid, sorbic acid, dehydroacetic acid, triclosan, benzyl alcohol, chlorophenesin, or for example.

In some embodiments, the preservative may comprise one or more of preservatives selected from the group consisting of organic acids, parabens, formaldehyde donors, phenol derivatives, quaternary ammoniums, alcohols, isothiazolinones, and combinations thereof. Preservatives having antibacterial activity are optionally present in the cosmetic skin conditioning compositions of the present invention. Examples of organic acid preservatives include, but are not limited to, sodium benzoate, potassium sorbate, benzoic acid and dehydroacetic acid, sorbic acid, and combinations thereof. A preferred organic acid preservative system includes a mixture of sodium benzoate and potassium sorbate. Examples of paraben preservatives include, but are not limited to, alkyl para-hydroxybenzoates, wherein the alkyl radical has from 1, 2, 3, 4, 5 or 6 carbon atoms and for example, from 1 to 4 carbon atoms e.g., methyl para-hydroxybenzoate (methylparaben), ethyl para-hydroxybenzoate (ethylparaben), propyl para-hydroxybenzoate (propylparaben), butyl para-hydroxybenzoate (butylparaben) and isobutyl para-hydroxybenzoate (isobutylparaben). Examples of formaldehyde donor preservatives include, but are not limited to, 1,3-Dimethylol−5,5-dimethylhydantoin (DMDM hydantoin), imidazolidinyl urea, gluteraldehyde, and combinations thereof. Examples of quaternary ammonium preservatives include, but are not limited to, benzalkonium chloride, methene ammonium chloride, benzethonium chloride, and combinations thereof. Examples of alcohol preservatives include, but are not limited to, ethanol, benzyl alcohol, dichlorobenzyl alcohol, phenoxyethanol, and combinations thereof. Examples of isothiazolone preservatives include, but are not limited to, methylchloroisothiazolinone, methylisothiazolinone, and combinations thereof.

Other suitable preservatives include, but are not limited to, chloracetamide, triclosan and iodopropynyl butylcarbamate, pyridine derivatives (e.g., pyrithione and zinc pyrithione), chlorphenesin, phenyl mercuric salts, phenoxyethanol, and other known preservative systems.

In some embodiments, the preservative includes one or more preservatives, the one or combination present at a concentration, by weight of about 0.001% to about 5%, or alternatively about 0.05% to about 2.5% or alternatively about 0.1% to about 2.0%, based upon weight of the cosmetic skin conditioning composition.

Thus, any one of or a combination of preservatives, when present, may be present, by weight, based on the total weight of the cosmetic skin conditioning composition, is from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 1.0, 1.1, 1.2, 1.3, 1.4, 1.5, 1.6, 1.7, 1.8, 1.9, 2, 3, 4, to about 5 weight percent, including increments and ranges therein and there between.

Other Optional components

In accordance with the disclosure, various non-limiting embodiments of the cosmetic skin conditioning composition may optionally include one or more other additives or actives(optional components), selected from, for example, actives such as acetyl trifluoromethylphenyl valylglycine; a humectant in addition to any glycol, for example caprylyl glycol, squalane, sugars, such as, glucose, xylitol, maltitol, sorbitol, sucrose pentaerythritol, inositol, pyrrolidone carboxylic acid, lactic acid, lithium chloride, acetamide MEA, dicyanamide, hyaluronic acid, aloe vera, honey, and seaweed extract; preservative and anti-microbial components (such as those listed above), particularly, for example, salicylic acid, and phenoxyethanol; phenolic compounds, such as chalcones, flavones, flavanones, flavanols, flavonols, dihydroflavonols, isoflavonoids, neoflavonoids, catechins, anthocyanidins, tannins, lignans, aurones, stilbenoids, curcuminoids, alkylphenols, betacyanins, capsacinoids, hydroxybenzoketones, methoxyphenols, naphthoquinones, and phenolic terpenes, resveratrol, curcumin, pinoresinol, ferulic acid, hydroxytyrosol, cinnamic acid, caffeic acid, p-coumaric acid, baicalin (Scutellaria Baicalensis root extract), pine bark extract (Pinus Pinaster bark/bud extract), ellagic acid; and vitamins and vitamin derivatives, such as calcium pantothenate, tocopherol and ascorbic acid; fillers such as clays, talc, organic thickeners with for instance, anionic, cationic, nonionic, and amphoteric polymeric associative thickeners and combinations thereof; penetrants; sequestrants; fragrances; dispersants; film-forming agents; ceramides; opacifiers; organic or inorganic sunscreen actives and SPF boosters; and combinations thereof.

Although the aforementioned optional components are given as an example, it will be appreciated that other optional components compatible with cosmetic applications known in the art may be used.

In some embodiments, one or more optional components, alone or in combination, can be present in the cosmetic skin conditioning composition according to the disclosure from about 0.05% to about 50% by weight, or from about 1% to about 30%, or from about 1.5% to about 20%, and from about 5% to about 15%, or from about 0.05% to about 2.5% by weight, or from about 0.1% to about 2%, or from about 0.25% to about 5%, and from about 0.5% to about 10%, or any suitable combination, sub-combination, range, or sub-range thereof by weight, based on the weight of the cosmetic skin conditioning composition.

In some embodiments, one or more other components, such as preservatives, vitamins, and the like, alone or in combination, can be present in the cosmetic skin conditioning composition according to the disclosure from about 0.05 to about 50% by weight, or from about 0.05% to about 25% by weight, or from about 0.1 to about 10%, or from about 0.25% to about 5%, and from about 0.5 to about 3.5%, or any suitable combination, sub-combination, range, or sub-range thereof by weight, based on the weight of the cosmetic skin conditioning composition.

In some exemplary embodiments, preservatives may include salicylic acid, and phenoxyethanol.

Thus, one or a combination of optional components may be present, by weight, based on the total weight of the cosmetic skin conditioning composition, or from about 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 to about 50 weight percent, including increments and ranges therein and there between.

Methods of Use

In accordance with the disclosure, the invention also provides methods of use of the cosmetic skin conditioning composition.

In some embodiments, a method of use includes application of the cosmetic skin conditioning composition as a leave on for providing moisture retention benefits.

In some embodiments, a method of use includes application of the cosmetic skin conditioning composition as a leave on, followed by application, of a complementary composition, such as but not limited to a complementary composition containing one or more actives that can compliment or supplement the benefits of the cosmetic skin conditioning composition. In some embodiments after application of the complementary composition, both compositions are either left on the skin or my be removed by rinsing. In some embodiments, the application of one or both may include use of an implement such as a brush.

### EXAMPLES

### Example 1

### Raw Materials

The examples below according to the invention are given by way of illustration and without a limiting nature. The names are the chemical name or the INCI name. The amounts of active ingredients are given therein as % by weight based on raw material compositions comprising 100% of the listed ingredient, unless otherwise mentioned. Compositions and systems as described in the representative embodiments herein are selected from commercially available materials, including, as shown in the exemplified embodiments herein below, for example: Urea, glycine, serine, Latic acid, sodium lactate, triacetin, and triethyl citrate.

TABLE 2
Raw Materials

| RM | PERCENT ACTIVE |
|---|---|
| INGREDIENT | (IF < 100%) |
| Urea | |
| Glycine | |
| Serine | |
| Latic Acid | 90 |
| Sodium Lactate | 60 |
| Triacetin | |
| Triethyl Citrate | |

### Example 2

### Inventive Compositions and Systems

Five exemplary embodiments of the inventive composition are shown in Table 3.

TABLE 3
Inventive Composition

| INGREDIENT | INV #1 | INV #2 | INV #3 | INV #4 | INV #5 |
|---|---|---|---|---|---|
| UREA | 40 | 40 | 30 | 30 | 30 |
| SODIUM LACTATE | 2.5 | 2.5 | 2.5 | 2.5 | 2.5 |
| SALICYLIC ACID | 0.45 | 0.45 | 0.45 | 0.45 | 0.45 |
| LACTIC ACID | 0.5 | 0.5 | 0.5 | 0.5 | 0.5 |
| AMMONIUM LACTATE | | 2.5 | | | |
| TRIACETIN | 0.4 | 0.7 | 0.7 | 0.7 | 0.7 |
| GLYCINE | 5 | 5 | 5 | 5 | 5 |
| ACETYL | | | | | 2 |
| TRIFLUORO- | | | | | |
| METHYLPHENYL | | | | | |
| VALYLGLYCINE | | | | | |
| PROPYLENE GLYCOL | 7 | 4.5 | 7 | 7 | 7 |
| GLYCERIN | | | | 3.5 | 3 |
| CAPRYLYL GLYCOL | | | 0.3 | 0.3 | 0.3 |
| TRIETHANOLAMINE | 0.9 | 0.9 | 0.9 | 0.9 | 0.9 |
| PHENOXYETHANOL | | 0.7 | 0.7 | 0.7 | 0.7 |
| AQUA | 43.25 | 42.25 | 51.95 | 48.45 | 46.95 |

### Example 3

### Effect of Amino Acid on pH

In studies, the effect of amino acid (glycine) on pH was evaluated in compositions with 20% urea and with 40% urea. Stability of pH was not possible at either amount of urea when the amount of amino acid (glycine) was below 5%. The pH was stabilized in a range from about 5.5 to about 6.5 above 5% and up to about 9% amino acid (glycine). At and above 10% amino acid (glycine), the pH was stable in a range from about 5.5 to about 6.5 but the cosmetic skin conditioning composition was unstable as evidenced by crystal formation.

### Example 4

### Effect of Buffer and Amino Acid (Glycine or Serine at 5%) on pH and Stability

In studies, the effect of buffer on pH and stability was evaluated in compositions with 20% urea and with 40% urea after a period of 4 weeks at 45° C. and at 4° C. In all cases, in the presence of glycine at 5%, with and without buffer, the pH remained stable in a range from between 5.5 and 6.5, with overall better performance with buffer (a change of less than 10% over the time period). Similar results were obtained with serine, in a higher average range from about 7 to just above pH 7.5. In the absence of any amino acid, the pH increased from an average just above 7.5 at the beginning of the time period to greater than 8.5 (a change of more than 20% to almost 50% over the time period), with slightly better performance with buffer (a change more than 20% to about 35% over the time period).

### Example 5

### Effect of 5% Proline vs 3% and 5% Glycine on pH at Varying Temperatures Over Time

In studies, using a base composition according to INV#1, the amino acid was substituted to evaluate the effect of % Proline vs 3% and 5% Glycine on pH at varying temperatures over time in (with 40% urea). In all cases the starting pH was about 5.5. At 4° C. no meaningful effect was observed over 8 weeks, and at 55° C. a similar increase in pH was observed (increase to between 7 and 7.4) within one week. At temperatures of 25° C., 37° C. and 45° C., glycine was the most effective at stabilizing the pH as compared with proline, and 5% glycine stabilized the pH more effectively than the 3% glycine. At all temperatures, each of proline and 5% glycine maintained the pH at or below 7 through a period of 8 weeks.

### Example 6

### Effect of Triacetin

In studies, using a base composition according to INV#1 (40% urea, 5% glycine, 0.7% triacetin, buffer), triacetin was varied to evaluate its effect on pH and stability. Triacetin was varied at 0.1%, 0.4% and 0.7% and substituted with 1.2% and 2.4% triethyl citrate, and each sample was evaluated at 4° C., 25° C., 37° C. and 45° C. In all examples, the results were comparable, with all samples showing a nominal change in pH at 4° C., 25° C. and 37° C., and all samples showing a change of about 10% to about 15% in pH at 45° C.

### Example 7

### Effect of Glycols on pH and Stability

In studies, the effect of glycols on the pH and stability (from crystallization) of the composition of INV #1 with 40% urea and 5% amino acid (glycine) was varied in glycol content and glycol composition (glycerin and/or propylene glycol). Two samples of each formulation were tested, and the formulations included from 0% to 7% glycol (glycerin or propylene glycol), and glycerin:propylene glycol in ratios from 4:1 to 1:4.

In the pH study, all samples were maintained for 8 weeks, at 25° C. and 45° C. In all cases, the pH remained stable in a range from about 5.9 to about 6.9.

In the stability study, only the formulations that contained only propylene glycol (at 3.5% and 7%) were free from crystal formation at both 4° C. at week 3, and after a freeze thaw cycle, as shown in Table 4.

TABLE 4

| INGREDIENT | VAR A | VAR B | VAR C | VAR D | VAR E | VAR F | VAR G | INV 1 | VAR I | VAR J |
|---|---|---|---|---|---|---|---|---|---|---|
| WATER | 42.25 | 42.25 | 42.25 | 42.25 | 42.25 | 42.25 | 42.25 | 42.25 | 42.25 | 42.25 |
| WATER | 1.17 | 2.33 | 0.00 | 4.67 | 3.50 | 7.00 | 1.17 | 0.00 | 3.50 | 0.00 |
| PROPYLENE | 1.17 | 2.33 | 0.00 | 1.17 | 0.00 | 0.00 | 4.67 | 7.00 | 3.50 | 3.50 |
| GLYCOL | | | | | | | | | | |
| GLYCERIN | 4.67 | 2.33 | 7.00 | 1.17 | 3.50 | 0.00 | 1.17 | 0.00 | 0.00 | 3.50 |
| WEEK 3; 4° C. (2 | 100% | 50% | 50% | 50% | 100% | 100% | 100% | 0% | 0% | 0% |
| samples, 0, 50% | | | | | | | | | | |
| or 100% crystal) | | | | | | | | | | |
| **FT; −20~20° C. | crystal | Crystal | Free at | Free at | Crystal | Free at | Crystal | Free at | Free at | Crystal |
| | | | 24 h | 16 hr | | 16 hr | | 16 hr | 16 hr | |

**Freeze thaw

### Example 6

### Effect of Urea vs Hydroxyethyl Urea on Peel Efficacy Using a Hydroxy Acid

In studies, the effect of hydroxyethyl urea (HEU) was compared with urea in a base composition according to INV#1 (40% urea, 5% glycine, 0.7% triacetin, buffer) in which the amount and form of urea was varied. The test compositions included inventive compositions: INV #1 (40% urea), INV #1 (a) having 23% urea, INV #1 (b) having 23% HEU, and INV #1 (c) having 40% HEU. Studies were conducted with each of the tested compositions to evaluate effect of the compositions on the stratum corneum.

In Vitro Stratum Corneum Comparison of A T using differential scanning calorimetry (DSC) is used to characterize the stability of a protein or other biomolecule directly in its native form. It does this by measuring the heat change associated with the molecule's thermal denaturation when heated at a constant rate. Compositions were evaluated on SC samples according to the following procedure. Stratum corneum samples were equilibrated at 75% overnight. Inventive and Comparative Compositions and control treatments applied on SC surface (0.1 g/sample) for 15 min in 75% relative humidity (RH). The SC surface was wiped clean and rinsed with DI water. Allow to dry in 75% RH overnight. The DSC profile was measured from 20° C. to 120° C. at 5° C./min.

As an initial step, each test composition was applied first to skin without rinsing, followed by application of a hydroxy-acid based peel composition in a moisturizer form. The studies revealed that increased amounts of each of urea and hydroxyethyl urea resulted in increased desquamation as compared to use of the peel moisture alone, the desquamation evidenced by stratum corneum disorganization. Surprisingly, the compositions with urea outperformed those with HEU, and in particular, the 23% urea form performed as well as the 40% HEU form.

While illustrative embodiments have been illustrated and described, it will be appreciated that various changes can be made therein without departing from the spirit and scope of the invention.

The articles “a” and “an,” as used herein, mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“At least one,” as used herein, means one or more and thus includes individual components as well as mixtures/combinations.

The transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinarily associated with the specified material(s). The term “consisting essentially of” limits the scope of a claim to the specified elements, steps or material(s) and those that do not materially affect the basic and novel characteristic(s) of the claimed invention. All materials and methods described herein that embody the present invention can, in alternate embodiments, be more specifically defined by any of the transitional terms “comprising,” “consisting essentially of,” and “consisting of.” Further, It should be noted that for purposes of this disclosure, terminology such as “upper,” “lower,” “vertical,” “horizontal,” “inwardly,” “outwardly,” “inner,” “outer,” “front,” “rear,” etc., should be construed as descriptive and not limiting the scope of the claimed subject matter. Further, the use of “including,” “comprising,” or “having” and variations thereof herein is meant to encompass the items listed thereafter and equivalents thereof as well as additional items. Unless limited otherwise, the terms “connected,” “coupled,” and “mounted” and variations thereof herein are used broadly and encompass direct and indirect connections, couplings, and mountings. The term “about” means plus or minus 5% of the stated value.

Other than in the operating examples, or where otherwise indicated, all numbers expressing quantities of ingredients and/or reaction conditions modified by the term “about” are to be understood as being within 10% of the indicated number (e.g. “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used, and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A skin conditioning composition, comprising:
a) urea or a derivative thereof, present in an amount from about 20% to about 40%, by weight, based on the weight of the composition;
b) at least one amino acid;
c) at least one of triacetin or triethyl citrate;
d) optionally, a buffer system having a pH in a range from about 4.0 to about 6.0, wherein the pH of the skin conditioning composition is less than about 7.

2. The skin conditioning composition according to claim 1, wherein the composition is formulated as a leave-on product.

3. The skin conditioning composition according to claim 1, wherein the at least one amino acid is soluble in water at a pH from about 4 to about 6 and is selected from the group consisting of glycine, proline, serine, cysteine and combinations thereof.

4. The skin conditioning composition according to claim 1, wherein the urea or a derivative thereof, is present in an amount from greater than about 20% to about 40%, the at least one amino acid is present in an amount from about 5% to about 9%, and the at least one of triacetin or triethyl citrate is present in an amount from about 0.2% to about 2%, all amounts by weight, based on the weight of the composition.

5. The skin conditioning composition according to claim 1, wherein
a) the at least one amino acid is selected from the group consisting of glycine, proline, serine, cysteine and combinations thereof and is present in an amount from about 5% to about 9%, by weight, based on the weight of the composition;
b) the at least one of triacetin or triethyl citrate is present in an amount a range that includes about 0.2% to about 2%, by weight, based on the weight of the composition;
c) the skin conditioning composition comprises a buffer system comprising one or a combination of components selected from the group consisting of citric acid, sodium citrate, sodium lactate, lactic acid, and combinations thereof,
wherein the skin conditioning composition demonstrates structural and pH stability for periods of up to about 8 weeks under temperature conditions that range from 4° C. up to 45° C. and through freeze thaw cycles from −20° C. through 20° C., characterized as the absence of any or sustained visible crystallization and the stabilization of pH at or under a pH of about 7.

6. The skin conditioning composition according to claim 5, further comprising at least one pH adjusting reagent comprising triethanolamine.

7. The skin conditioning composition according to claim 1, wherein the urea or a derivative thereof, is present in an amount from greater than about 20% to about 40%, by weight, based on the weight of the composition, and further comprising at least one glycol selected from the group consisting of propylene glycol, glycerin, and combinations thereof.

8. The skin conditioning composition according to claim 7, wherein the skin conditioning composition comprises propylene glycol and excludes glycerin.

9. A skin conditioning composition, comprising:
a) urea present from greater than 20% to about 40%;
b) at least one amino acid, the at least one amino acid comprising glycine present from about 5% to about 9%;
c) at least one of triacetin or triethyl citrate, present from about 0.2% to about 2%;
d) a buffer system having a pH in a range from about 4.0 to about 6.0, the buffer system comprising one or a combination of components selected from the group consisting of citric acid, sodium citrate, sodium lactate, lactic acid, and combinations thereof;
e) at least one pH adjusting reagent, comprising triethanolamine; and
f) at least one glycol selected from the group consisting of propylene glycol, glycerin, and combinations thereof,
wherein all amounts are by weight, based on the weight of the composition and wherein the skin conditioning composition demonstrates structural and pH stability for periods of up to about 8 weeks under temperature conditions that range from 4° C. up to 45° C. and through freeze thaw cycles from −20° C. through 20° C., characterized as the absence of any or sustained visible crystallization and the stabilization of pH at or under a pH of about 7.

10. The skin conditioning composition according to claim 9, wherein the skin conditioning composition comprises propylene glycol and excludes glycerin.

11. The skin conditioning composition according to claim 9, wherein the buffer system comprises one or a combination of components selected from the group consisting of citric acid, sodium citrate, sodium lactate, lactic acid, and combinations thereof.

12. The skin conditioning composition according to claim 9, wherein the buffer system comprises sodium lactate and lactic acid.

13. A skin conditioning composition, comprising: a carrier system comprising a water phase, the water phase comprising urea or a derivative thereof, present in an amount from about 20% to about 40%, by weight, based on the weight of the composition; at least one amino acid; at least one of triacetin or triethyl citrate; and, optionally, a buffer system having a pH in a range from about 4.0 to about 6.0, wherein the pH of the skin conditioning composition is less than about 7.

14. The skin conditioning composition according to claim 13, wherein the skin conditioning composition demonstrates structural and pH stability for periods of up to about 8 weeks under temperature conditions that range from 4° C. up to 45° C. and through freeze thaw cycles from −20° C. through 20° C., characterized as the absence of any or sustained visible crystallization and the stabilization of pH at or under a pH of about 7.

15. The skin conditioning composition according to claim 13, wherein the carrier system further comprises one more additional ingredients, each present in the water phase or in a second phase.

16. The skin conditioning composition according to claim 15, wherein the second phase is an oily phase.

17. The skin conditioning composition according to claim 13, wherein the one or more additional ingredients includes one or more of an additional ingredient selected from the group consisting of a skin active compound, a humectant in addition to any glycol, an oil, a surfactant, a thickener, a preservative, and combinations thereof.

18. The skin conditioning composition according to claim 13, wherein the cosmetic composition is one of a single phase solution comprising the water phase, a multi-phase solution comprising at least one additional phase, or an emulsion comprising at least one oily phase.

19. The skin conditioning composition according to claim 13, wherein the skin conditioning composition is a product for keratinous tissue selected from a toner, a micellar water, a cream, a lotion, a cleanser, or a make-up remover.

20. The skin conditioning composition according to claim 13, wherein the skin conditioning composition is a leave-on formulation.
